(19) United States
(12) Patent Application Publication
(10) Pub. No.: US 2026/0363999 A1
(43) Pub. Date: Nov. 19, 2026

(54) ORAL CARE COMPOSITION COMPRISING A NATURAL COLORANT

(71) Applicant: The Procter & Gamble Company, Cincinnati, OH (US)

(72) Inventors: Paul Albert SAGEL, Maineville, OH (US); Andrew Frederic GROTH, Mason, OH (US); James Albert BERTA, West Chester, OH (US)

(21) Appl. No.: 19/662,130

(22) Filed: Apr. 29, 2026

Related U.S. Application Data
(60) Provisional application No. 63/797,151, filed on Apr. 30, 2025.

(51) Int. Cl. A61K 8/9789; A61K 8/49; A61K 8/9794; A61K 8/98; A61Q 11/00

(52) U.S. Cl. CPC A61K 8/9789; A61K 8/492; A61K 8/9794; A61K 8/987; A61Q 11/00; A61K 2800/28; A61K 2800/30; A61K 2800/87

(57) ABSTRACT

An oral care composition comprising a natural colorant. The oral care composition may be free of or substantially free of synthetic colorants.

### CROSS REFERENCE TO RELATED APPLICATION

This application claims the benefit of U.S. Provisional Application No. 63/797,151, filed Apr. 30, 2025, the substance of which is incorporated herein by reference.

### TECHNICAL FIELD

The disclosure relates generally to oral care products that contain a natural colorant.

### BACKGROUND

Oral care products, like many other consumer products, commonly include a colorant to provide the product with a desirable color. Many of the colorants used in consumer products today are synthetic pigments, dyes or lakes. Relative to natural colorants, synthetic colorants are generally cheaper to make, more stable, and produce more vibrant colors. However, consumer groups and government agencies have recently expressed concern over the safety, environmental friendliness and sustainability of certain synthetic colorants. For example, the U.S. Food and Drug Administration recently banned the use of FD&C Red #3 (a.k.a. erythrosine) in food and ingested drugs due to safety concerns.

The use of synthetic colorants in oral care products such as dentifrice compositions, mouth rinses, dental floss and tooth whitening compositions can be especially concerning to consumers and manufacturers of such products because the composition or ingredients in the composition may be ingested and/or irritate the oral mucosa. While the synthetic colorants approved for use in oral care products may still be safe for use, at least some consumers prefer products that are free of synthetic colorants (e.g., “clean label” products)

Accordingly, it would be desirable to provide oral care products that contain natural colorants. It would also be desirable to provide oral care products that are free of, or essentially free of, synthetic colorants.

### SUMMARY

Disclosed herein is an oral care composition comprising a natural colorant and is, optionally, free of or substantially free of synthetic colorants.

### DETAILED DESCRIPTION

Synthetic dyes can be particularly suitable for use in oral care composition because they can be tailored to be inert, which helps provide stability and color-fastness. Natural dyes, on the other hand, may exhibit instability (e.g., precipitate or migrate) or a lack of color fastness at certain pH (e.g., acidic). Surprisingly, it has now been found that by specifically tailoring the ingredients and/or the ratios of certain ingredients in the composition, natural colorants can used to provide desirable color and/or stability.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition and are based on level of the subject ingredient, which does not include carriers or by-products that may be included in commercially available materials, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25 °C and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### DEFINITIONS

“About” modifies a particular value by referring to a range of plus or minus 20% or less of the stated value (e.g., plus or minus 15% or less, 10% or less, or even 5% or less).

“Dentifrice composition” means a tooth or subgingival-paste, gel, or liquid formulation used to clean teeth or deliver a tooth and/or gum health benefit. The dentifrice composition may be a single-phase composition, a multi-phase composition, or a combination of two or more separate dentifrice compositions. The dentifrice composition may be in any desired form, such as deep striped, surface striped, multilayered, a gel surrounding a paste, or any combination thereof. Each dentifrice composition in a dentifrice comprising two or more separate dentifrice compositions may be contained in a physically separated compartment of a dispenser and dispensed, for example, side-by-side.

“Color stable” means the composition and/or colorant passes the Accelerated Appearance Stability test and/or the Photostability test, as described in more detail below.

“Dye” refers to a colorant formed by dissolving a solid (e.g., powder) or liquid (e.g., plant juice) in a suitable solvent (e.g., water, oil or alcohol).

“Effective amount” means an amount of a compound or ingredient (e.g., colorant) that is sufficient to result in a desired effect.

“Lake” refers to a colorant that is combined with an inert binder (e.g., potassium aluminum sulfate (“alum”)) to render it insoluble in water and oil and then dispersed in a suitable liquid.

“Natural colorant” refers to colorants that are obtained directly from a plant, animal or mineral. Natural colorants can also include colorants obtained directly from a plant, animal or mineral and then physically and/or chemically modified (e.g., to improve purity and/or pH stability) without altering the essential structure or function of the colorant.

“Oral care composition” means a composition that is intended for use in the oral cavity to deliver an oral health benefit but is not intentionally swallowed. Examples of oral care compositions include toothpaste, mouthwash, and dental floss coatings.

“Oral care product” means an oral care composition disposed in one or more packages (e.g., a primary package and a secondary package) suitable for marketing to a consumer.

“Pigment” refers to a colorant (e.g., powder) that is generally insoluble in oil and water, but can be dispersed in a suitable liquid.

“Substantially free” means that no more than 1% (e.g., no more than 0.5%, 0.1%, 0.05%, 0.01%, or 0.001%), of an indicated material in a composition, by total weight of such composition.

“Synthetic colorant” refers to colorants synthesized from petroleum- or coal-based compounds (e.g., coal tar). Some non-limiting examples of synthetic colorants include Citrus Red #2, Orange B, FD&C Red #3, FD&C Green #3, FD&C Red #40, FD&C Yellow #5, FD&C Yellow #6, FD&C Blue #1, and FD&C Blue #2.

### Oral Care Composition

The oral care compositions herein may be in any suitable form, such as a solid, liquid, powder, paste, or combinations thereof. The oral care composition can be a dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces, such as tooth whitening strips. Some non-limiting examples of emulsion compositions are disclosed in U.S. Pat. Nos. 11,147,753 and 11,096,874. Some non-limiting examples of unit-dose compositions are disclosed in U.S. Publication No. 2019/0343732.

The oral care compositions herein may include a variety of active ingredients, inactive ingredients, and optional ingredients such as, for example, hops, dicarboxylic acids, tin ion sources, calcium ion sources, fluoride ion sources, zinc ion sources, polyphosphates, humectants, surfactants, water, polyols, preservatives, thickeners, pH adjusters, colorants, abrasives, chelants, and surfactants.

In some instances, the oral care composition may be provided in a transparent or translucent package (e.g., primary and/or secondary packaging). In such oral care products, it can be important for the oral care composition to be color stable. Some natural colorants are known to exhibit undesirable photo instability when exposed to visible and/or UV light. This photo instability can manifest as a reduction in color intensity and/or a change in color (e.g., green to brown).

### Natural Colorant

The oral care products herein include a natural colorant. Some non-limiting examples of sources of natural colorants obtained from plants (or algae) include pomegranate, strawberry, raspberry, blackberry, blueberry, elderberry, cherry, grapes, beet, carrot (orange and purple), apple, banana peel, custard seed, safflower, yellow bell pepper, yellow tomatoe, banana pepper, yellow squash, pumpkin, malt, burnt sugar, red radish, paprika, Mahonia aquifolium (a.k.a. Oregon grape), turmeric, saffron, Camellia sinensis (a.k.a. green tea) (e.g., for matcha colorant), spinach, parsley, purple cabbage, cucumber, celery, seaweed, chard, mustard greens, ube (a.k.a. purple sweet potato), acai, coffee, cocoa, cinnamon, dogwood, Indigofera tinctoria (e.g., for indigo colorant), Persicaria tinctoria (e.g., for Japanese indigo colorant), Indigofera suffruticosa (e.g., for Maya blue colorant), Isatis inctoria (a.k.a. woad), Haematoxylum campechianum (a.k.a. logwood), Iris, cornflower, hibiscus, Clitoria ternatea (a.k.a. butterfly blue pea), hyacinth, red algae, blue-green algae (e.g., for blue spirulina colorant), black cocoa, Bixa orellana (a.k.a. achiote) (e.g., for annatto colorant), Moringa oleifera (a.k.a. moringa tree), Genipa americana (a.k.a. genip tree) (e.g., for huito fruit) corn starch, grasses (e.g., barley grass and wheat grass), alfalfa, and combinations thereof.

Some non-limiting examples of natural colorants obtained from animals include squid ink and carmine (red pigment derived from Dactylopius coccus), and combinations thereof.

Some non-limiting examples of natural colorants obtained from minerals include copper compounds (e.g., copper salts (e.g., copper chloride), copper oxides, copper glycinate, and copper gluconate), metal salts (e.g., chloride) and oxides (e.g., salts and oxides of iron, aluminum, tin, titanium, copper, magnesium, silver, cobalt, nickel, chromium, calcium, and combinations of these such as cobalt aluminum oxide), calcium pyrophosphate, kaolin, boron nitride, lazurite, ferric ferrocyanide (Prussian blue), calcium peroxide, barium carbonate, barium oxide, barium sulfate, fused silica, vermiculite, zeolite, mixtures of zinc sulfide and barium sulfate (lithopone),

Other sources of natural colorants can include sand, shells, insoluble starch-mineral complexes, emulsified fats and combinations thereof.

Some non-limiting examples of natural colorant compounds include lutein, riboflavin, lycopene, chlorophyll, anthocyanins, carotene (e.g., beta carotene and its complexes), beta-apo 8, curcumin, phycocyanin (blue spirulina), and combinations of these.

Some particularly suitable examples of natural colorants that may be suitable for use herein include carmine, curcumin/turmeric, blue butterfly pea, purple butterfly pea, huito, beet juice, chlorophyll and combinations thereof. Commercially available sources of natural colorants include those sold under the BRIGHT'N Pure™ and BRIGHT'N Free™ brands from IFF, those sold under the PearlEdge™ brand from Archer Daniels Midlant (ADM), and SensiOp™ brand from Sensient Beauty.

Table 1 shows some examples of colors that can be obtained from natural colorants. Color may be defined by any suitable means known in the art. Some non-limiting examples of color models include Red-Green-Blue (RGB) color space; Cyan, Magenta, Yellow and Black (CMYK) color space, CIELAB color space, Hue-Saturation-Lightness (HSL), and Hue-Saturation-Value (HSV). In some instances, it may be important to tailor the amount, type and/or ratio of the natural colorant(s) in the composition to provide a consumer preferred color in the oral care product.

TABLE 1
Natural Colorants

| Color | Source |
|---|---|
| Red | Beet, Pomegranate, Carmine, |
| | Cochineal extract |
| Pink | Raspberry, Anthorcyanins |
| Orange | Carrot, Paprika, Annato, Beta- |
| | carotene, Beta-apo 8 |
| Yellow | Turmeric, Saffron, Curcumin |
| Green | Chlorophyll, Matcha, Spinach, |
| | Parsley, copper chlorophyllin, |
| | Pandan |
| Blue | Purple cabbage, Spirulina, Huito, |
| | Butterfly pea, Copper compounds |
| | (e.g., copper salts and oxides) |
| Purple | Blueberry, Purple carrots, Ube, |
| | Acai, Elderberry, Tyrian Purple, |
| | Radish |
| Brown | Coffee, Cocoa, Cinnamon, Burnt |
| | Sugar, Caramels |
| Black | Activated charcoal, Squid ink, |
| | Black cocoa |
| White/Silver | Titanium dioxide, Corn starch, |
| | Calcium pyrophosphate, Boron |
| | nitride, kaolin |

The colorants herein can be obtained using conventional means known in the art. For example, a colorant may be obtained from a portion of a plant (e.g., peel, fruit, seed, stem, bark, leaves, roots, or a combination of these). In some instances, the plant or a portion thereof may be macerated, followed by a liquid-liquid extraction technique. In another example, a colorant may be obtained by boiling a portion or all of a plant and straining the liquid to obtain the colorant. In still another example, a plant or portion thereof may be subjected to a serum fraction technique, for example, as described in U.S. Pat. Nos. 7,442,391 and 7,537,791, to obtain the desired colorant compound(s). In a further example, the plant may be dried and ground into a powder to provide a pigment. It is to be appreciated that the method of obtaining the naturally derived colorant is not particularly limited, and the present application contemplates other methods that are not explicitly disclosed herein.

The colorant may be provided as an aqueous composition that includes more than 20% water and/or a water miscible carrier such as glycerin (e.g., more than 25%, 30%, 35%, 40%, 45%, 50%, 55%, 60%, 65%, 70%, or even 75% or more). In some embodiments, the colorant may be provided in the form of an anhydrous solution or a powder. The colorant may be provided as an emulsion, suspension, encapsulate, flake (e.g., colorant incorporated into a matrix of hydroxypropyl cellulose, microcrystalline cellulose and/or starch), wax (e.g., colorant incorporated into a wax matrix), prill (e.g., colorant incorporated into a matrix of solid polyethylene glycol), micronized powder, or combinations thereof.

The colorant should be stable in the oral care compositions herein and exhibit suitable color stability. For example, in some embodiments, the colorant should exhibit no more than a 40% change (e.g., no more than a 35%, 30%, 25%, 20%, 15%, 10%, 9, 5%, 4%, 3%, 2%, or 1% change, or even no change) in one or more of L*, a*, b*, hue angle or chroma according to the Colorimetry method hereinbelow.

### Tin

The oral care compositions of the present invention comprise tin, also referred to as “stannous” or “Sn” herein. The tin can be provided by a tin ion source. The tin ion source can be any suitable compound that can provide tin ions in an oral care composition and/or deliver tin ions to the oral cavity when the dentifrice composition is applied to the oral cavity. The tin ion source can comprise one or more tin containing compounds, such as stannous fluoride, stannous chloride, stannous bromide, stannous iodide, stannous oxide, stannous sulfate, stannous sulfide, stannic fluoride, stannic chloride, stannic bromide, stannic iodide, stannic sulfide, and/or mixtures thereof. Preferably, the tin ion source can comprise stannous fluoride, stannous chloride, and/or mixture thereof.

In some instances, it may be desirable to provide a mixed-dentate complex (i.e., a monodentate ligand and a polydentate ligand) as described in U.S. Pub. No. 2025/0009619 due to the unique properties of stannous ion in solution (tetrahedral bonding geometry with 2+ formal valence).

In some instances, it may be desirable to include hops beta acid, which acts as a special ligand for metal cations coordinating through a beta acid and the carbonyl on the non-prenyl functional group attached to the central ring. Coordination of Sn in the presence of other ligands by hops may increase the soluble Sn²⁺ in oral care compositions comprising both stannous and hops beta acids. The performance of such ternary systems is difficult to anticipate because of the myriad interactions that can exist in the formula.

The oral care compositions can comprise from about 0.0025% to about 5%, from about 0.2% to about 1%, from about 0.5% to about 1.5%, or from about 0.3% to about 0.6%, by weight of the oral care composition, of a tin ion source.

### Polyol

The oral care compositions may include a polyol (i.e., an organic compound with more than one hydroxyl functional groups) that can weakly associate, interact, or bond to tin ions while the oral care composition is stored prior to use. The polyol can be a sugar alcohol, a monosaccharide, a disaccharide, a polysaccharide, or a non-reducing sugar. Sugar alcohols are a class of polyols that can be obtained through the hydrogenation of sugar compounds with the formula (CHOH)ₙH₂, where n=4-6. Preferably, n is 5 and/or 6 because these compounds have been shown to unexpectedly interact with tin ion sources to create soluble complexes, as described herein.

The polyol can be glycerin, erythritol, xylitol, sorbitol, mannitol, butylene glycol, lactitol, galactitol, and/or combinations thereof. In some instances, the polyol can be a sugar alcohol with the formula (CHOH)ₙH₂, where n=4-6 (e.g., xylitol, sorbitol, galactitol, and/or mixtures thereof. The oral care composition may include 0.01% to about 70%, from about 5% to about 70%, from about 5% to about 50%, from about 10% to about 60%, or from about 20% to about 80%, by weight of the oral care composition, of a polyol.

### Abrasive

The oral care composition may include a calcium or silica abrasive to help remove surface stains from teeth. Calcium abrasives include any suitable abrasive calcium compounds that can provide calcium ions in an oral care composition and/or deliver calcium ions to the oral cavity when the oral care composition is applied to the oral cavity. Some non-limiting examples of calcium abrasives include calcium carbonate, precipitated calcium carbonate (PCC), ground calcium carbonate (GCC), chalk, dicalcium phosphate, calcium pyrophosphate, and/or combinations thereof. Some non-limiting examples of silica abrasives include silica gel (by itself, and of any structure), precipitated silica, amorphous precipitated silica (by itself, and of any structure), hydrated silica, and/or combinations thereof.

Other abrasives that may be suitable for use herein include bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins, and combinations thereof. T

The oral care composition can comprise from about 5% to about 70%, from about 10% to about 60%, from about 20% to about 50%, from about 25% to about 40%, or from about 1% to about 50% of an abrasive.

Alternatively, the oral care composition can be substantially free of, essentially free of, or free of abrasives (e.g., less than 5%, less than 1%, less than 0.5%, less than 0.1%, or 0%).

### Water

The oral care compositions herein may be anhydrous, low-water formulations, or high-water formulations. In total, the oral care composition may include 1% to 80% water (e.g., 5% to 75%, 10% to 70%, 15% to 60% or 20% to 50%).

pH

The oral care compositions herein may have a pH of 3.5 to 10.5 (e.g., 4.0-10.0, 4.5-9.5, 5.0-9.0, 5.5-8.5, or 6.0-8.0 or any combination of these ranges). The pH of a mouthrinse solution can be determined as the pH of the neat solution. The pH of a dentifrice composition can be determined as a slurry pH, which is the pH of a mixture of the dentifrice composition and water at a ratio of dentifrice to water of 1:3.

The oral care composition can comprise one or more buffering agents to adjust or maintain the pH of the composition. Some non-limiting examples of buffering agents include alkali metal hydroxides, carbonates, sesquicarbonates, borates, silicates, phosphates, imidazole, and mixtures thereof. Buffering agents that may be particularly suitable for use herein include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition may include one or more buffering agents at a level of from about 0.1% to about 30%, from about 1% to about 10%, or from about 1.5% to about 3%, by weight of the present composition.

### Anti-oxidants

The oral care composition herein may include an antioxidant such as, for example, butylated hydroxyanaline (BHA), butylates hydroxy tolulene (BHT), vitamins, pyrophosphates, radical scavangers (e.g., salicylic acid), sodium stannate.

### Zinc

The oral care composition may include zinc provided by a zinc ion source such as, for example, zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate zinc carbonate, or a combination of these.

The zinc ion source may be present at an amount of 0.01% to 5% (e.g., 0.2% to 1%, 0.5% to 1.5%, or 0.3% to 0.6%).

### Fluoride

The oral care composition may include fluoride provided by a fluoride ion source such as, for example, stannous fluoride, sodium fluoride, potassium fluoride, amine fluoride, sodium monofluorophosphate, zinc fluoride, and/or mixtures thereof. The fluoride ion source and the tin ion source can be the same compound (e.g., stannous fluoride). The fluoride ion source and the zinc ion source can be the same compound (e.g., zinc fluoride).

The fluoride ion source may be present in the total oral care composition at 0.0025% to 5% (e.g., 0.2% to 1%, 0.5% to 1.5%, or 0.3% to 0.6%, by weight of the oral care composition. The fluoride ion source should be present at an amount sufficient to provide 50 ppm to 3500 ppm, (e.g., 500 ppm to 3000 ppm) of free fluoride ions in the composition.

### Polyphosphates

The oral care composition a polyphosphate provided by a polyphosphate source.

Polyphosphates are a class of materials obtained by the dehydration and condensation of orthophosphate to yield linear and cyclic polyphosphates of varying chain lengths. Polyphosphate molecules are generally identified by an average number (n) of polyphosphate molecules, which is well-known.

Polyphosphates that may be suitable for use herein have the formula:

XO(XPO₃)ₙX,
Where:
   X is an alkali metal cation such as sodium, potassium, or ammonium, and
   n is 2 to 21.

Some non-limiting examples of polyphosphates that may be suitable for use herein include pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

When the composition comprises fluoride, it may be desirable to exclude alkaline earth metals such as calcium (e.g., calcium pyrophosphate) because they tend to form insoluble fluoride salts in aqueous solutions. Thus, the oral care compositions disclosed herein can be free of or substantially free of calcium pyrophosphate.

The polyphosphate source may be present at 0.01% to 15% (e.g., 0.1% to 10%, 0.5% to 5%, 1 to 20%, or 10% or less).

### Surfactants

The oral care composition may include one or more surfactants at a level of 0.01% to 15%, (e.g., 0.3% to 10% or 0.3% to 2.5%), by weight of the oral care composition, to impart suitable detersive and/or foaming properties. The surfactant may include an anionic surfactant, cationic surfactant, nonionic surfactant, zwitterionic surfactant, amphoteric surfactant and combinations thereof. Some non-limiting examples of amphoteric and zwitterionic surfactants that may be suitable for use herein include the betaine family of surfactants (e.g., cocamidopropyl betaine, lauramidopropyl betaine, lauryl betaine, palmityl betaine, and coco betaine).

Some non-limiting examples of anionic surfactants include sodium lauryl sulfate, sodium lauryl isethionate, sodium lauroyl methyl isethionate, sodium cocoyl glutamate, sodium dodecyl benzene sulfonate, the sodium, potassium, and ethanolamine salts of N-lauroyl, N-myristoyl, and N-palmitoyl sarcosine,

Some non-limiting examples of non-ionic surfactants include polyethylene oxide condensates of alkyl phenols (e.g., nonylphenol ethylene oxide condensates), polyoxyethylene, sorbitan monostearate, sorbitan isostearate and sorbitan laurate.

Some non-limiting examples of cationic surfactants include quaternary ammonium compounds such as cetylpyridinium chloride, cetyl pyridinium fluoride, tetradecylpyridinium chloride, N-tetradecyl-4-ethyl pyridinium chloride, domiphen bromide, benzalkonium chloride, benzethonium chloride, methyl benzethonium chloride, dodecyl trimethyl ammonium bromide, dodecyl dimethyl(2-phenoxyethyl) ammonium bromide, benzyl dimethoxystearyl ammonium chloride, quaternized 5-amino-1,3-bis(2-ethyl-hexyl)-5-methyl hexa hydropyrimidine, lauryl trimethylammonium chloride, cocoalkyl trimethylammonium chloride, cetyl trimethylammonium bromide, di-isobutylphenoxyethyl-dimethylbenzylammonium chloride, dodecyl trimethyl ammonium bromide, are exemplary of typical quaternary ammonium antimicrobial agents. Particularly suitable examples are cetylpyridinium, or tetradecylpyridinium halide salts (i.e., chloride, bromide, fluoride and iodide).

Additional examples of surfactants that may be suitable for use herein are disclosed in U.S. Publication No. 2025/0009619.

### Thickening Agents

The oral care composition may include a thickening agent at a level of 0.01% to 15% (e.g., 0.1% to 10%, 0.2% to 5%, or 0.5% to 2%). Thickening agents are generally useful for providing a suitable structure and/or stabilize the composition (e.g., inhibit or prevent phase separation in a toothpaste). Suitable thickening agents include polysaccharides, polymers, and/or silica thickeners.

Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Some non-limiting examples of polymers that may be suitable for use herein include polyethylene glycol (PEG), polyvinylpyrrolidone (PVP), polyacrylic acid, polymers derived from at least one acrylic acid monomer, copolymers of maleic anhydride and methyl vinyl ether, crosslinked polyacrylic acid polymers.

Some non-limiting examples of suitable inorganic thickening agents include colloidal magnesium aluminum silicate and silica thickeners (e.g., ZEODENT® 165 brand amorphous precipitated silica, ZEODENT® 153, 163, and 167 brand silica products, ZEOFREE® 177 and 265 brand silica products, and AEROSIL® brand fumed silicas, all from Evonik).

### Chelant

The oral care composition may include a chelant capable of binding to the divalent metal ions present in the composition. When tin ions are present, it may be desirable to select a chelant that forms a water-soluble complex with the tin ions. When the oral care composition includes silica abrasives and tin ions, it may be desirable to select a chelant that reversibly binds the tin ions to prevent the tin ions from irreversibly binding to the surface of silica particles.

Chelants suitable for use herein have a weight average molecular weight of less than 1000 Daltons. Some non-limiting examples of chelants include C₂-C₆ dicarboxylic and tricarboxylic acids, such as succinic acid, malic acid, tartaric acid and citric acid; C₃-C₆ monocarboxylic acids substituted with hydroxyl, such as gluconic acid; picolinic acid; amino acids such as glycine; phytic acid, salts thereof and mixtures thereof. A particularly suitable chelant is sodium gluconate or a salt of citric acid.

The oral care composition can comprise one or more chelants each at a level of from about 0.01% to about 5%, from about 0.2% to about 2%, from about 0.5% to about 1.5%, or from about 1% to about 3%, by weight of the oral care composition. Alternatively, the oral care composition can be substantially free of or free of a chelant because the oral care compositions of the present invention comprises a calcium abrasive instead of a silica abrasive. Additionally, the oral care composition can comprise one or more chelants each at a level of less than about 1%, less than about 0.5%, less than about 0.1%, or less than about 0.01%, by weight of the oral care composition.

### Other Ingredients

The oral care composition can comprise a variety of other ingredients, such as flavoring agents, sweeteners, colorants, preservatives, buffering agents, or other ingredients suitable for use in oral care compositions, as described below.

In some embodiments, the oral care composition may include 0.001% to 5% of a flavoring agent. Some non-limiting examples of flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants such as paramenthan and carboxyamides (e.g., N-ethyl-p-menthan-3-carboxamide and N-(Ethoxycarbonylmethyl)-3-p-menthanecarboxamide), may also be part of the flavor system.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

The oral care composition may include synthetic colorants that have been approved by the appropriate regulatory bodies (e.g., U.S. FDA) and those listed in the European Food and Pharmaceutical Directives. Some nonlimiting examples include FD&C and D&C dyes.

Preservatives such as methylparaben, propylparaben, benzoic acid, and sodium benzoate can be added to the oral care compositions in safe and effective amounts to prevent bacterial growth.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

Humulus lupulus (Hops)

The oral care compositions herein may include a hops compound from Formula I and/or Formula II shown below. Formula I are hops alpha acids and Formula II are hops beta acids.

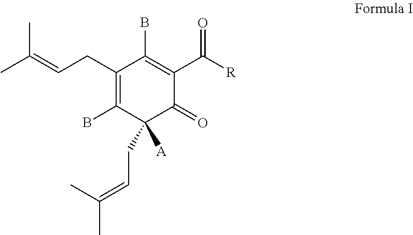

### Where:

A is an acidic hydroxyl functional group in the alpha position,
B are acidic hydroxyl functional groups in the beta position, and
R is an alkyl.

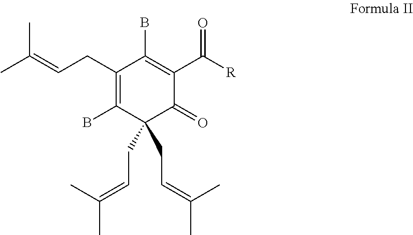

### Where:

B are an acidic hydroxyl functional groups in the beta position, and
R is an alkyl.

The hops compound can be provided by any suitable source, such as an extract from Humulus lupulus, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Some non-limiting examples of hops alpha acids (generically shown in Formula I) include humulone, adhumulone, cohumulone, posthumulone, prehumulone, and/or mixtures thereof. Some non-limiting examples of hops iso-alpha acids include cis-isohumulone and/or trans-isohumulone. Some non-limiting examples of hops beta acids include lupulone, adlupulone, colupulone, and/or mixtures thereof.

Hops acids exhibit antibacterial activity and thus may be suitable for use as an anticaries agent in oral care compositions. While hops alpha acids demonstrates some antibacterial activity, it also has a bitter taste, which can make it unsuitable for use in oral care compositions. In contrast, hops beta acids exhibit higher antibacterial and/or anticaries activity, relative to hops alpha acid, and is less bitter tasting. Thus, a hops extract with a higher proportion of beta acids to alpha acids than normally found in nature may be particularly suitable for use herein.

A natural hops source generally comprises about 2% to 12% hops beta acids, depending on the variety of hops. The hops extract desired herein should comprise at least 35 wt % hops beta acids (e.g., at least 40% or 45%) up to 90%, 95% or even up to 99%.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise from about 2% to about 12%, by weight of the hops source, of hops alpha acids. Hops extracts used in other contexts, such as in the brewing of beer, can comprise from about 15% to about 35%, by weight of the extract, of hops alpha acids. The hops extract desired herein can comprise less than about 10%, less than about 5%, less than about 1%, or less than about 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

The hops compound can be combined with or free from an extract from another plant, such as a species from genus Magnolia, Garcinia mangostana L., or Zizyphus joazeiro. The hops compounds can be combined with or free from a nonionic halogenated diphenyl ether, such as triclosan.

The hops beta acid may be present at 0.01% to 10% (e.g., 0.05% to 10%, 0.1% to 8%, 0.2% to 6%, 0.5% to 5%, 0.2% to 4%, 0.25% to 3%, or 0.5 to 2%).

### Dicarboxylic Acid

The oral care composition herein may include a dicarboxylic acid. Some non-limiting examples of dicarboxylic acids that may be suitable for use herein include oxalic acid, malic acid, maleic acid, malonic acid, methylmalonic acid, dimethylmalonic acid, dihydroxymalonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, tartaric acid, tartronic acid, phthalic acid, terephthalic acid, mesoxalic acid, fumaric acid, glutaric acid, their salts, and combinations thereof. Particularly suitable examples of dicarboxylic acid compounds include malonic acid, methylmalonic acid, tartronic acid, malic acid, dimethylmalonic acid, mesoxalic acid, dihydroxymalonic acid, and oxalic acid, due to their unexpectedly high whitening benefit. While not wishing to be bound by theory, it is believed that particular dicarboxylic acid compounds have an unexpectedly high affinity to certain cationic crosslinking agents typically found in the colored matrix on the oral hard tissue surfaces, thereby resulting in the removal of stain from the surface.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, K, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Antibacterial Agents

The oral care composition may include an antibacterial agent to provide antibacterial activity in the oral cavity. Suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof.

The oral care composition may include 0.01% to 10% (e.g., 1% to 5%, or 0.5% to 15%) of an antibacterial agent

### Bioactive Materials

The oral care composition may include bioactive materials for remineralizing a tooth. Some non-limiting examples of bioactive materials include bioactive glasses (e.g., Novamin™ brand bioactive glass from GSK and Recaldent™ brand bioactive glass from Cadbury Schweppes) hydroxyapatite, calcium phosphate, and amino acids (e.g., arginine, citrulline, glycine, lysine, or histidine, or combinations thereof). Suitable examples of compositions comprising arginine are found in U.S. Pat. Nos. 4,154,813 and 5,762,911.

The oral care composition can comprise from about 0.01% to about 20%, from about 0.1% to about 10%, or from about 1% to about 10% of a bioactive material by weight of the oral care composition.

### Potassium

The oral care composition may include a potassium ion source selected from one or more potassium containing compounds such as potassium nitrate, potassium fluoride, potassium chloride, and combinations thereof.

The potassium ion source may be present in the oral care composition at 0.01% to 10% (e.g., 0.2% to 1%, 0.4% to 1%, or 0.3% to 0.6%). Alternatively, the oral care composition can be essentially free of, substantially free of, or free of potassium

### Prenylated Flavonoids

The oral care composition may include a prenylated flavonoid. Flavonoids are a group of natural substances found in a wide range of fruits, vegetables, grains, bark, roots, stems, flowers, tea, and wine that are known to exhibit a variety of beneficial effects (e.g., antioxidative, anti-inflammatory, antimutagenic, anticarcinogenic, and antibacterial benefits). A prenylated flavonoid is a flavonoid that includes at least one prenyl functional group (3-methylbut-2-en-1-yl) that is known to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane.

Increasing the lipophilicity to increase penetration into the cell membrane can be a double-edged sword because the prenylated flavonoid will tend towards insolubility at high Log P values (high lipophilicity). Log P can be an important indicator of antibacterial efficacy and suitable prenylated flavonoids will have a log P of at least 2 (e.g., at least 4, 2 to 10, 4 to 10, or 4 to 7). A more detailed description of prenylated flavonoids suitable for use herein can be found in U.S. Publication No. 2025/0009619.

Prenylated flavonoids may be present in the oral care composition at 0.001% or more (e.g., 0.001% to 5%, 0.01% to 2%, 0.1% to 2%, or about 0.05%).

### Whitening Agent

The oral care composition may include 0.1% to 10% (e.g., 0.2% to 5%, 1% to 5%, or 1% to 15%) of a compound suitable for whitening teeth. Some non-limiting examples of whitening agents that may be suitable for use herein include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, and mixtures thereof.

### Humectant

The oral care composition can comprise one or more humectants, have low levels of a humectant, be essentially free of, be substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Oral Care Composition Forms

Suitable compositions forms include emulsion compositions such as those of U.S. Pat. No. 11,147,753, unit-dose compositions such as those of U.S. Patent Application Publication No. 2019/0343732, leave-on oral care compositions, jammed emulsions such as those of U.S. Pat. No. 11,096,874, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

In some instances, it may be desirable to protect the colorant from interacting with other ingredients in the composition, including any carrier (e.g., water) that may be present to improve color vibrancy and/or stability. For example, the colorant may be encapsulated, flaked, waxed, or prilled to physically isolate it from the rest of the composition. In some instances, the colorant may be chemically modified to render it less reactive or even inert.

### Methods

The oral care compositions herein can lead to oral health benefits, such as the treatment, reduction, and/or prevention of caries, cavities, gingivitis, and/or combinations thereof and/or the whitening of teeth, removing stain from teeth, and/or preventing the accumulation of stain from teeth when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure.

The user can also be instructed to rinse with a mouthwash and/or mouth rinse composition after the completion of the brush procedure or instead of the brush procedure. The user can be instructed to swish the oral care composition thoroughly for at least 30 seconds, at least one minute, at least 90 seconds, or at least two minutes at least once, at least twice, or at least three times per day. The user can also be instructed to expectorate the oral care composition after the completion of the procedure.

The oral care compositions according to embodiments of the present invention can be used in the treatment, reduction, and/or prevention of caries, cavities, gingivitis, and/or combinations thereof. The oral care compositions according to embodiments of the present invention can be used to provide a whitening benefit, such as the whitening of teeth, removing stain from teeth, and/or preventing the accumulation of stain from teeth. For example, as described herein, hops beta acid can be useful as an antigingivitis agent. Thus, the addition of hops to any oral care composition can provide antigingivitis protection.

The oral care composition can include primary packaging, such as a tube, bottle, and/or tub. The primary package can be placed within secondary package, such as a carton, shrink wrap, or the like. Instructions for use of the oral care composition can be printed on the primary package and/or the secondary package. The scope of the method is intended to include instructions provided by a manufacturer, distributor, and/or producer of the oral care composition.

The user can be instructed to dispense a dose of toothpaste from a tube and apply at least some of the toothpaste onto a toothbrush. The portion of toothpaste applied to the toothbrush can be of any suitable shape, such as strip, a pea-sized amount, or various other shapes that would fit onto any mechanical and/or manual brush head. The user can be instructed to apply a strip of the toothpaste that is at least about 1 inch, at least about 0.5 inch, at least 1 inch, and/or at least 0.5 inch long to the bristles of a toothbrush, such as soft-bristled toothbrush.

The user can be instructed to brush their teeth for at least about 30 seconds, at least about 1 minute, at least about 90 seconds, at least about 2 minutes, at least 30 seconds, at least 1 minute, at least 90 seconds, and/or at least 2 minutes.

The user can be instructed to brush their teeth after each meal. The user can be instructed to brush their teeth at least once per day, at least twice per day, and/or at least three times per day. The user can be instructed to brush their teeth no more than three times a day, such as to prevent Sn staining. The user can be instructed to brush their teeth in the morning and/or in the evening prior to sleeping.

The user can be instructed to not swallow the toothpaste composition due to the inclusion of ingredients that are not suitable for ingestion, such as fluoride. However, in the case of an oral care composition comprising hops, but free of fluoride, the user may not need to be instructed to not swallow the toothpaste. The user may be instructed to expectorate (or spit out) the toothpaste composition after the cessation of the brushing cycle.

If the oral care composition is a mouth rinse, the user can be instructed to perform one or more of the following: dispense the mouth rinse from a bottle containing the mouth rinse, use the mouth rinse at least once a day (e.g., twice a day or three times a day), use the mouth rinse composition after the use of toothpaste and/or floss, swish the rinse in the oral cavity for a period of time (e.g., at least 30 seconds, 1 minute, 90 seconds or even 2 minutes or more), use 5 mL to 50 mL of mouth rinse (e.g., 10 mL to 40 mL, 15 mL to 35 mL, 20 mL to 30 mL), not swallow the mouth rinse, expectorate (or spit out) the mouth rinse after cessation of the rinse cycle.

The usage instructions for the oral care composition, such as for a toothpaste composition and/or a mouth rinse composition, can vary based on age. For example, adults and children that are at least 6 or at least 2 can have one usage instruction while children under 6 or under 2 can have a second usage instruction.

Additionally, the oral care composition, as described herein, can be used to reduce the number and/or intensity of white spots on teeth, which can be attributable to caries presence within the oral cavity. Or the oral care composition, as described herein, can be used to reduce the redness, puffiness, tenderness, and/or swollenness of gums at the gumline immediately adjacent the surfaces of the teeth, which can be attributable to gingivitis presence within the oral cavity.

### EXAMPLES

### Accelerated Appearance Stability Test

This method can be used to evaluate whether a composition or colorant is likely to exhibit an undesirable color change when exposed to the environmental conditions typically experienced by an oral care composition during manufacturing, transportation and storage. The test results are based on a PASS/FAIL outcome, where a PASS indicates acceptable color stability and FAIL indicates an undesirable color change (i.e., unacceptable color instability).

The test is conducted by aging a test composition at 40 °C and 75% relative humidity for 1 month and comparing the appearance of the test composition to control composition, which is aged for 1 month a 5 °C. Color is evaluated by an expert with 20/20 vision under normal daytime lighting conditions at a distance of 30 cm to 60 cm. Any undesirable change in color or color intensity or type (e.g., green to brown, blue to gray) results in a FAIL.

### Photostability Test

This method can be used to evaluate whether a composition or dye exhibits an undesirable color change when exposed to light. As manufacturers move more towards recycling friendly packaging such as 100% HDPE and open walled packages, the products contained in the packages may be exposed to higher levels of light, which can degrade or destabilize photosensitive materials like natural colorants. Thus, it can be important for natural colorants used in oral care composition to have suitable photostability.

This test is conducted on samples at 25 °C and 50% relative humidity according to the Photostability Testing of New Drug Substances and Products Q1B, step 4 version dated Nov. 6, 1996, published by the International Conference on Harmonization of Technical Requirements for Registration of Pharmaceuticals for Human Use (ICHQ1B). Higher viscosity compositions such as toothpaste are stored in a white HDPE tube. Lower viscosity composition such as mouth rinse composition compositions are stored in a glass vial. The test samples are exposed to 1200 klux-hr of white visible light followed by 200 W-hr/m² of UV light. After exposure the samples are judged to determine if an undesirable color change has occurred and, if so, identified as a FAIL. If no undesirable color change has occurred, then the test sample is identified as a PASS.

### Formulations

This example demonstrates the color stability of certain natural colorants in an oral care composition. Natural colorants that include “ADM” in the name were obtained from the Archer-Daniels-Midland Company, Chicago, Illinois. Natural colorants that include “IFF” in the name were obtained from International Flavors & Fragrances, Inc., New York, New York. Natural colorants that include “Sensient” in the name were obtained from Sensient Technologies Corporation, Milwaukee, Wisconsin. Natural colorants that include “California Natural Color” in the name were obtained from California Natural Color company, Fresno, California. Composition formulations are shown in Tables 2-6.

The compositions were prepared by adding the indicated colorant to a premade base composition (i.e., toothpaste or mouthrinse), which was made using conventional methods known to those skilled in the art. The colorant is mixed homogeneously into the oral care composition (e.g., speedmixer for toothpaste compositions and magnetic stir bar for mouth rinse compositions). All ingredients are listed in weight percent, based on the weight of the composition. Control compositions were made with synthetic colorants or titanium dioxide.

The toothpaste example compositions included 3 different types of base compositions: sodium fluoride (Ex. 1-20), sodium fluoride+pyrophosphate (Ex. 21-39), and stannous fluoride (Ex. 40-81). The formulations for the examples are provided below in Table 2. The colorant was added to the base toothpaste composition at 1%. The mouthrinse example compositions included 2 different base compositions: Cetylpyridinium chloride (CPC) (Ex. 82-95) and CPC+fluoride (Ex. 96-118). The formulations are provided below in Table 3. The colorants used in the compositions are listed Tables 4-8 below.

TABLE 2
Toothpaste compositions

| | Ex. 1-20 | Ex. 21-39 | Ex. 40-57 | Ex. 58-68 | Ex. 69-79 | Ex. 80 | Ex. 81 |
|---|---|---|---|---|---|---|---|
| Sorbitol, 70% solution | 67.4080 | 34.4000 | 49.5000 | 46.0000 | 46.0000 | 46.0000 | 46.0000 |
| Water | QS | QS | QS | QS | QS | QS | QS |
| Stannous fluoride | — | — | 0.4540 | 0.4540 | 0.4540 | 0.4540 | 0.4540 |
| Stannous chloride | — | — | 0.5619 | 0.5619 | 0.5619 | 0.5619 | 0.5619 |
| dihydrate (10% silica) | | | | | | | |
| Sodium Fluoride | 0.2430 | 0.2430 | — | — | — | — | — |
| Carboxymethylcellulose | 0.7500 | — | — | — | — | — | — |
| Sodium Acid | — | 4.2100 | — | — | — | — | — |
| Pyrophosphate Anhydrous | | | | | | | |
| Sodium hydroxide, 50% | — | 2.1770 | 0.8700 | 0.8700 | 0.8700 | 0.8700 | 0.8700 |
| solution | | | | | | | |
| Sodium citrate dihydrate | — | — | 1.0500 | 1.0500 | 1.0500 | 1.0500 | 1.0500 |
| Citric acid anhydrous | — | — | — | — | — | 0.4000 | 0.4000 |
| Zinc citrate dihydrate | — | — | 0.5330 | 0.5330 | 0.5330 | 0.5330 | 0.5330 |
| Sodium gluconate | — | — | 1.3000 | 1.3000 | 1.3000 | 1.3000 | 1.3000 |
| Poloxamer 407 | — | 0.2000 | — | — | — | — | — |
| Saccharin | 0.3500 | 0.4500 | 0.5000 | 0.4000 | 0.4000 | 0.4000 | 0.4000 |
| Sucralose, 25% solution | — | — | 0.2000 | 0.2000 | 0.2000 | 0.2000 | 0.2000 |
| Carrageenan (10% silica) | — | — | 1.4444 | 1.4444 | 1.4444 | 1.4444 | 1.4444 |
| Xanthan gum | — | 0.6000 | 0.7000 | 0.7000 | 0.7000 | 0.7000 | 0.7000 |
| Carbomer | 0.3000 | — | — | — | — | — | — |
| Silica | 15.0000 | 24.5000 | 17.5000 | 17.5000 | 17.5000 | 17.5000 | 17.5000 |
| Sodium Lauryl Sulfate, | 4.0000 | 6.0000 | 5.0000 | 5.0000 | 5.0000 | 5.0000 | 5.0000 |
| 29% solution | | | | | | | |
| Cocamidopropyl betaine, | — | 0.6000 | 2.0000 | 2.0000 | 2.0000 | 2.0000 | 2.0000 |
| 30% solution | | | | | | | |
| Sodium phosphate tribasic | 1.1000 | — | — | — | — | — | — |
| dodecahydrate | | | | | | | |
| Sodium phosphate | 0.4190 | — | — | — | — | — | — |
| monobasic monohydrate | | | | | | | |
| Flavor | 0.4500 | 1.4000 | 0.5000 | 1.2250 | 1.2250 | 1.2250 | 1.2250 |
| colorant | 1.0000 | 1.0000 | 1.0000 | 0.0200 | 0.1000 | 0.0200 | 0.1000 |
| total | 100.0000 | 100.0000 | 100.0000 | 100.0000 | 100.0000 | 100.0000 | 100.0000 |
| pH | 7.5 | 7.5 | 6.5 | 6.5 | 6.5 | 4.5 | 4.5 |

TABLE 3
Mouthrinse compositions

| | Ex. 82-95 | Ex. 96-118 |
|---|---|---|
| Glycerin | 5.0000 | 5.0000 |
| Water | QS | QS |
| Cetylpyridinium chloride | 0.0740 | 0.0450 |
| Sodium Fluoride | | 0.0219 |
| Saccharin | 0.0300 | 0.0250 |
| Sucralose | 0.0200 | 0.0150 |
| Benzoic acid | 0.0045 | 0.0045 |
| Sodium benzoate | 0.0537 | 0.0537 |
| Poloxamer 407 | 0.0600 | 0.0800 |
| Phosphoric acid 85% solution | — | 0.0583 |
| Sodium phosphate | — | 0.0010 |
| dibasic heptahydrate | | |
| Flavor | 0.1700 | 0.1700 |
| total | 100.0000 | 100.0000 |
| pH | 5.5 | 3.5 |

The oral care compositions were tested in the Accelerated Appearance Stability test and Photostability test. FD&C Blue 1, FD&C Green 3, FD&C Red 40, D&C Red 33, Sensient Green Shade 6631 (a blend of FD&C colors), and a green colored mixture of FD&C Blue 1 and FD&C Yellow 5 were used as controls across the various tests and compositions. A summary of the results is provided in Tables 4-8. The weight % of colorant in the mouthrinse composition is provided in Tables 7 and 8.

TABLE 4
NaF toothpaste results

| Exam- | | | Appearance | Photo- |
|---|---|---|---|---|
| ple | Colorant | Color | Stability | stability |
| Ex. 1 | ADM/WILD Flavors | Dark Blue/ | PASS | PASS |
| | Blue Color LWS VF | Purple | | |
| Ex. 2 | ADM/WILD Flavors | Purple | PASS | — |
| | Purple Color | | | |
| | LWS VFB | | | |
| Ex. 3 | ADM/WILD Flavors | Purple | PASS | PASS |
| | Purple Color | | | |
| | DWS HJB | | | |
| Ex. 4 | IFF Bright'n | Blue | FAIL | — |
| | Blue Spirulina | | | |
| Ex. 5 | Sensient Natural | Blue | PASS | PASS |
| | Blue Powder (Blue | | | |
| | Butterfly Pea Extract) | | | |
| Ex. 6 | FD&C Blue 1 1.1% | Blue | PASS | — |
| | solution (control) | | | |
| Ex. 7 | ADM/WILD | Green | PASS | PASS |
| | Flavors Green Color | | | |
| | LWS | | | |
| Ex. 8 | ADM/WILD | Green | PASS | FAIL |
| | Flavors Green Color | | | |
| | LWS VT | | | |
| Ex. 9 | Green mixture 1.1% | Green | PASS | — |
| | solution (55% Blue 1 & | | | |
| | 45% Yellow 5) (control) | | | |
| Ex. 10 | ADM/WILD Flavors | Red | FAIL | — |
| | CFN Red Color | | | |
| | LWS B | | | |
| Ex. 11 | ADM/WILD Flavors | Red | FAIL | — |
| | CFN Red Color | | | |
| | DWS BCNB | | | |
| Ex. 12 | ADM/WILD Flavors | Red | FAIL | — |
| | CFN Red Color | | | |
| | LWS BA | | | |
| Ex. 13 | IFF Bright'n | Orange/ | FAIL | — |
| | Yellow Annatto | Red | | |
| Ex. 14 | IFF Bright'n Red | Red | PASS | PASS |
| | Carmine 4 LWS | | | |
| Ex. 15 | IFF Red Radish Juice | Red | PASS | — |
| Ex. 16 | IFF Black Carrot Juice | Pink | FAIL | — |
| Ex. 17 | Sensient Bright | Red | PASS | PASS |
| | Red Powder | | | |
| Ex. 18 | FD&C Red 40 1% | Red | PASS | — |
| | solution (control) | | | |
| Ex. 19 | ADM/WILD Clear | Dark | PASS | PASS |
| | Beta-Carotene emulsion | Yellow/ | | |
| | | Orange | | |
| Ex. 20 | IFF Bright'n Yellow | Yellow | — | PASS |
| | Curcumin 15% | | | |
| | (Turmeric) | | | |

TABLE 5
NaF + pyrophosphate toothpaste results

| Exam- | | | Appearance | Photo- |
|---|---|---|---|---|
| ple | Colorant | Color | Stability | stability |
| Ex. 21 | ADM/WILD Flavors | Dark | PASS | PASS |
| | Blue Color LWS VF | Blue/ | | |
| | | Purple | | |
| Ex. 22 | IFF Bright'n Blue Spirulina | Blue | FAIL | — |
| Ex. 23 | ADM/WILD | Purple | PASS | — |
| | Flavors Purple Color | | | |
| | LWS VFB | | | |
| Ex. 24 | ADM/WILD | Purple | PASS | PASS |
| | Flavors Purple Color | | | |
| | DWS HJB | | | |
| Ex. 25 | Sensient Natural | Blue | FAIL | PASS |
| | Blue Powder (Blue | | | |
| | Butterfly Pea Extract) | | | |
| Ex. 26 | FD&C Blue 1 1.1% | Blue | PASS | — |
| | solution (control) | | | |
| Ex. 27 | ADM/WILD | Green | PASS | PASS |
| | Flavors Green Color | | | |
| | LWS | | | |
| Ex. 28 | ADM/WILD | Green | PASS | PASS |
| | Flavors Green Color | | | |
| | LWS VT | | | |
| Ex. 29 | Green mixture 1.1% | Green | PASS | — |
| | solution (55% Blue | | | |
| | 1 & 45% Yellow 5) | | | |
| | (control) | | | |
| Ex. 30 | ADM/WILD Flavors | Red | FAIL | — |
| | CFN Red Color | | | |
| | LWS B | | | |
| Ex. 31 | ADM/WILD Flavors | Red | FAIL | — |
| | CFN Red Color | | | |
| | DWS BCNB | | | |
| Ex. 32 | ADM/WILD Flavors | Red | FAIL | — |
| | CFN Red Color | | | |
| | LWS BA | | | |
| Ex. 33 | IFF Bright'n Red | Red | PASS | PASS |
| | Carmine 4 LWS | | | |
| Ex. 34 | IFF Red Radish Juice | Red | FAIL | — |
| Ex. 35 | Sensient Bright Red Powder | Red | PASS | PASS |
| Ex. 36 | IFF Black Carrot Juice | Pink | FAIL | — |
| Ex. 37 | FD&C Red 40 | Red | PASS | — |
| | 1% solution (control) | | | |
| Ex. 38 | ADM/WILD | Dark | PASS | PASS |
| | Clear Beta-Carotene | Yellow/ | | |
| | emulsion | Orange | | |
| Ex. 39 | IFF Bright'n Yellow | Yellow | PASS | PASS |
| | Curcumin 15% | | | |
| | (Turmeric) | | | |

TABLE 6
SnF₂ toothpaste results

| Exam- | | | Appearance | Photo- |
|---|---|---|---|---|
| ple | Colorant | Color | Stability | stability |
| Ex. 40 | ADM/WILD Flavors | Dark | FAIL | PASS |
| | | Blue/ | | |
| | Blue Color LWS VF | Purple | | |
| Ex. 41 | ADM/WILD Flavors | Purple | FAIL | — |
| | Purple Color | | | |
| | LWS VFB | | | |
| Ex. 42 | ADM/WILD | Purple | FAIL | PASS |
| | Flavors Purple Color | | | |
| | DWS HJB | | | |
| Ex. 43 | Sensient Natural | Blue | FAIL | PASS |
| | Blue Powder (Blue | | | |
| | Butterfly Pea Extract) | | | |
| Ex. 44 | FD&C Blue 1 1.1% | Blue | PASS | — |
| | solution (control) | | | |
| Ex. 45 | ADM/WILD | Green | PASS | PASS |
| | Flavors Green Color | | | |
| | LWS | | | |
| Ex. 46 | ADM/WILD | Green | PASS | PASS |
| | Flavors Green Color | | | |
| | LWS VT | | | |
| Ex. 47 | Sensient Green | Green | PASS | — |
| | Shade 6631 1.1% | | | |
| | solution (control) | | | |
| Ex. 48 | ADM/WILD Flavors | Red | FAIL | — |
| | CFN Red Color | | | |
| | LWS B | | | |
| Ex. 49 | ADM/WILD Flavors | Red | PASS | — |
| | CFN Red Color | | | |
| | DWS BCNB | | | |
| Ex. 50 | ADM/WILD Flavors | Red | FAIL | — |
| | CFN Red Color | | | |
| | LWS BA | | | |
| Ex. 51 | IFF Bright'n Red | Red | PASS | PASS |
| | Carmine 4 LWS | | | |
| Ex. 52 | IFF Red Radish Juice | Red | FAIL | |
| Ex. 53 | Sensient Bright Red Powder | Red | PASS | PASS |
| Ex. 54 | IFF Black Carrot Juice | Pink | PASS | PASS |
| Ex. 55 | FD&C Red 40 1% | Red | FAIL | — |
| | solution (control) | | | |
| Ex. 56 | IFF Bright'n Yellow | Yellow | PASS | PASS |
| | Curcumin 15% | | | |
| | (Turmeric) | | | |
| Ex. 57 | ADM/WILD Clear | Dark | PASS | PASS |
| | Beta-Carotene | Yellow/ | | |
| | emulsion | Orange | | |
| Ex. 58 | FD&C Blue 1 1.1% | Blue | PASS | — |
| | solution (control) | | | |
| Ex. 59 | IFF Spirulina LOD | Blue | FAIL | — |
| Ex. 60 | IFF Spirulina LWS | Blue | FAIL | — |
| Ex. 61 | IFF Copper | Green | PASS | — |
| | Chlorophyllin 10 LWS-AS | | | |
| Ex. 62 | IFF Copper | Green | PASS | — |
| | chlorophyll 10 OS | | | |
| Ex. 63 | IFF Red Radish | Pink/ | FAIL | — |
| | Anthocyanins LWS-D | Purple | | |
| Ex. 64 | IFF Carmine 4 LWS | Pink/Red | PASS | — |
| Ex. 65 | IFF Curcumin 5LWS | Yellow | PASS | — |
| Ex. 66 | IFF Annatto | Orange | PASS | — |
| Ex. 67 | IFF Beta-carotene 1.3LWS | Yellow | PASS | — |
| Ex. 68 | IFF Raspberry 724 Powder | Pink/Red | FAIL | — |
| Ex. 69 | FD&C Blue 1 1.1% | Blue | PASS | — |
| | solution (control) | | | |
| Ex. 70 | IFF Spirulina LOD | Blue | FAIL | — |
| Ex. 71 | IFF Spirulina LWS | Blue | FAIL | — |
| Ex. 72 | IFF Copper | Green | PASS | — |
| | Chlorophyllin 10 LWS-AS | | | |
| Ex. 73 | IFF Copper | Green | PASS | — |
| | chlorophyll 10 OS | | | |
| Ex. 74 | IFF Red Radish | Pink/ | FAIL | — |
| | Anthocyanins LWS-D | Purple | | |
| Ex. 75 | IFF Carmine 4 LWS | Pink/Red | PASS | — |
| Ex. 76 | IFF Curcumin 5LWS | Yellow | PASS | — |
| Ex. 77 | IFF Annatto | Orange | PASS | — |
| Ex. 78 | IFF Beta-carotene 1.3LWS | Yellow | PASS | — |
| Ex. 79 | IFF Raspberry 724 Powder | Pink/Red | FAIL | — |
| Ex. 80 | IFF Raspberry 724 | Pink/Red | FAIL | — |
| | Powder + Citric | | | |
| | Acid | | | |
| Ex. 81 | IFF Raspberry 724 | Pink/Red | FAIL | — |
| | Powder + Citric | | | |
| | Acid | | | |

TABLE 7
CPC mouthrinse results

| Exam- | | | | Appearance | Photo- |
|---|---|---|---|---|---|
| ple | Colorant | wt % | Color | Stability | stability |
| Ex. 82 | ADM/WILD | 0.0400 | Dark | PASS | Marginal- |
| | Flavors Blue | | Blue/ | | fades |
| | Color LWS VF | | Purple | | |
| Ex. 83 | ADM/WILD | 0.0399 | Purple | PASS | FAIL |
| | Flavors Purple | | | | |
| | Color DWS HJB | | | | |
| Ex. 84 | ADM/WILD | 0.2975 | Purple | PASS | — |
| | Flavors Purple | | | | |
| | Color LWS VFB | | | | |
| Ex. 85 | FD&C Blue 1 | 0.5000 | Blue | PASS | PASS |
| | 1.1% solution | | | | |
| | (control) | | | | |
| Ex. 86 | ADM/WILD | 0.0489 | Green | PASS | FAIL |
| | Flavors Green | | | | |
| | Color LWS | | | | |
| Ex. 87 | FD&C Green 3 | 0.2520 | Green | PASS | PASS |
| | 1% solution | | | | |
| | (control) | | | | |
| Ex. 88 | IFF Bright'n | 0.3665 | Red | PASS | PASS |
| | Red Carmine 4 | | | | |
| | LWS | | | | |
| Ex. 89 | IFF Black | 0.0200 | Pink | FAIL | FAIL |
| | Carrot Juice | | | | |
| Ex. 90 | ADM/WILD | 0.3400 | Red | FAIL | — |
| | Flavors CFN Red | | | | |
| | Color LWS BA | | | | |
| Ex. 91 | D&C Red 33 | 0.7447 | Red | PASS | PASS |
| | 1% solution | | | | |
| | (control) | | | | |
| Ex. 92 | IFF Bright'n | 0.4952 | Yellow | PASS | PASS |
| | Yellow Curcumin | | | | |
| | 7% (Turmeric) | | | | |
| Ex. 93 | IFF Bright'n | 0.0079 | Yellow | PASS | FAIL |
| | Yellow Curcumin | | | | |
| | 15% (Turmeric) | | | | |
| Ex. 94 | IFF Bright'nYellow | 0.0320 | Yellow | PASS | PASS |
| | Beta-Carotene | | | | |
| | 1.3 LWS | | | | |
| Ex. 95 | IFF Paprika | 0.0100 | Orange | FAIL | FAIL |
| | 2000 CU LWS | | | | |

TABLE 8
CPC + fluoride mouthrinse results

| Exam- | | | | Appearance | Photo- |
|---|---|---|---|---|---|
| ple | Colorant | wt % | Color | Stability | stability |
| Ex. 96 | ADM/WILD | 0.1267 | Purple | PASS | — |
| | Flavors Purple | | | | |
| | Color LWS VFB | | | | |
| Ex. 97 | IFF Bright'n Blue | 0.1696 | Blue | FAIL | — |
| | Spirulina | | | | |
| Ex. 98 | Sensient Natural | 0.0360 | Blue | PASS | FAIL |
| | Blue Powder | | | | |
| | (Blue Butterfly | | | | |
| | Pea Extract) | | | | |
| Ex. 99 | California Natural | 0.0847 | Purple | PASS | — |
| | Color Pure Purple | | | | |
| | Butterfly Pea | | | | |
| Ex. 100 | ADM/WILD | 0.0421 | Dark | PASS | PASS |
| | Flavors Blue | | Blue/ | | |
| | Color LWS VF | | Purple | | |
| Ex. 101 | ADM/WILD | 0.0421 | Purple | PASS | PASS |
| | Flavors Purple | | | | |
| | Color DWS HJB | | | | |
| Ex. 102 | FD&C Blue 1 | 0.5384 | Blue | PASS | PASS |
| | 1.1% solution | | | | |
| | (control) | | | | |
| Ex. 103 | ADM/WILD | 0.0526 | Green | PASS | PASS |
| | Flavors Green | | | | |
| | Color LWS | | | | |
| Ex. 104 | ADM/WILD | 0.0189 | Green | PASS | FAIL |
| | Flavors Green | | | | |
| | Color LWS VT | | | | |
| Ex. 105 | ADM/WILD | 0.1274 | Red | FAIL | — |
| | Flavors CFN | | | | |
| | Red Color LWS B | | | | |
| Ex. 106 | ADM/WILD | 0.2127 | Red | FAIL | — |
| | Flavors CFN | | | | |
| | Red Color | | | | |
| | DWS BCNB | | | | |
| Ex. 107 | ADM/WILD | 0.1697 | Red | FAIL | — |
| | Flavors CFN Red | | | | |
| | Color LWS BA | | | | |
| Ex. 108 | IFF Red | 0.1703 | Red | FAIL | — |
| | Radish Juice | | | | |
| Ex. 109 | IFF Bright'n Red | 0.3748 | Red | PASS | PASS |
| | Carmine 4 LWS | | | | |
| Ex. 110 | Sensient Bright | 0.0316 | Red | PASS | PASS |
| | Red Powder | | | | |
| Ex. 111 | IFF Black | 0.0211 | Pink | FAIL | FAIL |
| | Carrot Juice | | | | |
| Ex. 112 | IFF Purple | 0.1699 | Pink | FAIL | — |
| | Sweet Potato | | | | |
| Ex. 113 | Sensient Suprared | 0.0422 | Dark | FAIL | — |
| | (beet juice) | | Red | | |
| Ex. 114 | IFF Bright'n | 0.1282 | Orange/ | PASS | — |
| | Yellow Annatto | | Red | | |
| Ex. 115 | IFF Bright'n | 0.0210 | Yellow | PASS | PASS |
| | Yellow Curcumin | | | | |
| | 15% (Turmeric) | | | | |
| Ex. 116 | IFF Bright'nYellow | 0.0316 | Yellow | PASS | PASS |
| | Beta-Carotene | | | | |
| | 1.3 LWS | | | | |
| Ex. 117 | IFF Paprika | 0.1277 | Orange | PASS | — |
| | 2000 CU LWS | | | | |
| Ex. 118 | IFF Bright'n | 0.1701 | Yellow | PASS | — |
| | Yellow Curcumin | | | | |
| | 7% (Turmeric) | | | | |

### Example Combinations

1. An oral care composition comprising: a natural colorant, wherein the composition is free of synthetic colorants.
2. The oral care composition of paragraph 1, wherein the composition is color stable.
3. The oral care composition of paragraph 1 or 2, wherein the natural colorant is obtained from chlorophyll, pandan, blue pea extract, purple pea extract, pomegranate, strawberry, raspberry, blackberry, blueberry, elderberry, cherry, grapes, beet, carrot, apple, banana peel, custard seed, safflower, yellow bell pepper, yellow tomatoe, banana pepper, yellow squash, pumpkin, malt, burnt sugar, red radish, paprika, Mahonia aquifolium, turmeric, saffron, Camellia sinensis, spinach, parsley, purple cabbage, cucumber, celery, seaweed, chard, mustard greens, ube, acai, coffee, cocoa, cinnamon, dogwood, Indigofera tinctoria, Persicaria tinctoria, Indigofera suffruticosa, Isatis inctoria, Haematoxylum campechianum, iris, cornflower, hibiscus, Clitoria ternatea, hyacinth, red algae, blue-green algae, black cocoa, Bixa orellana, Moringa oleifera, Genipa americana, corn starch, grass, alfalfa, squid ink, carmine, copper compounds, metal salts and oxides, calcium pyrophosphate, kaolin, boron nitride, lazurite, ferric ferrocyanide, calcium peroxide, barium carbonate, barium oxide, barium sulfate, fused silica, vermiculite, zeolite, zinc sulfide, barium sulfate, sand, shells, insoluble starch-mineral complexes, emulsified fats, and combinations thereof,
4. The oral care composition of paragraph 3, wherein the natural colorant is selected from carmine, curcumin/turmeric, blue butterfly pea, purple butterfly pea, huito, beet, chlorophyll, and combinations thereof.
5. The oral care composition of any preceding paragraph, further comprising an abrasive.
6. The oral care composition of paragraph 5, wherein the abrasive comprises a silica abrasive, a calcium abrasive or a combination thereof.
7. The oral care composition of paragraph 6, wherein the abrasive comprises calcium carbonate.
8. The oral care composition of any preceding paragraph, wherein the oral care composition is free of silica.
9. The oral care composition of any preceding paragraph, further comprising a fluoride ion source.
10. The oral care composition of any one of paragraphs 1 to 9, wherein the oral care composition is free of fluoride.
11. The oral care composition of any preceding paragraph, further comprising a metal ion source selected from tin, zinc, potassium, copper, manganese, magnesium, strontium, titanium, iron, molybdenum, boron, cesium, aluminum, indium, and combinations thereof.
12. The oral care composition of paragraph 11, further comprising a tin ion source selected from stannous fluoride, stannous chloride, or a combination thereof.
13. The oral care composition of any preceding paragraph, further comprising hops.
14. The oral care composition of paragraph 13, wherein the hops comprises a beta acid
15. The oral care composition of paragraph 14, wherein the hops beta acid is non-hydrogenated hops beta acid.
16. The oral care composition of any preceding paragraph, wherein the composition has a pH of about 3.5 to about 10.5.
17. The oral care composition of any preceding paragraph, further comprising a monodentate ligand and a polydentate ligand.
18. The oral care composition of paragraph 17, wherein the monodentate ligand comprises a carboxylic acid selected from aliphatic carboxylic acids, aromatic carboxylic acids, amino acids, alpha hydroxy acids, beta hydroxy acids, gamma hydroxy acids, sugar acids, salts of these, and combinations thereof.
19. The oral care composition of paragraph 17, wherein the polydentate ligand comprises a compound with at least two functional groups capable of chelating tin.
20. The oral care composition of paragraph 19, wherein the polydentate ligand is selected from oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, citric acid, isocictric acid, aconitic acid, propane-1,2,3-tricarboxylic acid, trimesic acid, pyrophosphate, tripolyphosphate, tetrapolyphosphate, hexametaphosphate, salts of these, and combinations thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising: a natural colorant, wherein the composition is free of synthetic colorants.

2. The oral care composition of claim 1, wherein the composition is color stable.

3. The oral care composition of claim 1, wherein the natural colorant is obtained from chlorophyll, pandan, blue pea extract, purple pea extract, pomegranate, strawberry, raspberry, blackberry, blueberry, elderberry, cherry, grapes, beet, carrot, apple, banana peel, custard seed, safflower, yellow bell pepper, yellow tomato, banana pepper, yellow squash, pumpkin, malt, burnt sugar, red radish, paprika, Mahonia aquifolium, turmeric, saffron, Camellia sinensis, spinach, parsley, purple cabbage, cucumber, celery, seaweed, chard, mustard greens, ube, acai, coffee, cocoa, cinnamon, dogwood, Indigofera tinctoria, Persicaria tinctoria, Indigofera suffruticosa, Isatis inctoria, Haematoxylum campechianum, iris, cornflower, hibiscus, Clitoria ternatea, hyacinth, red algae, blue-green algae, black cocoa, Bixa orellana, Moringa oleifera, Genipa americana, corn starch, grass, alfalfa, squid ink, carmine, copper compounds, metal salts and oxides, calcium pyrophosphate, kaolin, boron nitride, lazurite, ferric ferrocyanide, calcium peroxide, barium carbonate, barium oxide, barium sulfate, fused silica, vermiculite, zeolite, zinc sulfide, barium sulfate, sand, shells, insoluble starch-mineral complexes, emulsified fats, and combinations thereof.

4. The oral care composition of claim 3, wherein the natural colorant is selected from carmine, curcumin/turmeric, blue butterfly pea, purple butterfly pea, huito, beet, chlorophyll, and combinations thereof.

5. The oral care composition of claim 1, wherein the natural colorant is present at 0.0001% to 10 wt %, based on the weight of the composition.

6. The oral care composition of claim 1, further comprising an abrasive.

7. The oral care composition of claim 6, wherein the abrasive comprises a silica abrasive, a calcium abrasive or a combination thereof.

8. The oral care composition of claim 7, wherein the abrasive comprises calcium carbonate.

9. The oral care composition of claim 1, wherein the oral care composition is free of silica.

10. The oral care composition of claim 1, further comprising a fluoride ion source.

11. The oral care composition of claim 1, wherein the oral care composition is free of fluoride.

12. The oral care composition of claim 1, further comprising a metal ion source selected from tin, zinc, potassium, copper, manganese, magnesium, strontium, titanium, iron, molybdenum, boron, cesium, aluminum, indium, and combinations thereof.

13. The oral care composition of claim 11, further comprising a tin ion source selected from stannous fluoride, stannous chloride, or a combination thereof.

14. The oral care composition of claim 1, further comprising hops.

15. The oral care composition of claim 14, wherein the hops comprises a non-hydrogenated beta acid.

16. The oral care composition of claim 1, further comprising a monodentate ligand and a polydentate ligand.

17. The oral care composition of claim 17, wherein the monodentate ligand comprises a carboxylic acid selected from aliphatic carboxylic acids, aromatic carboxylic acids, amino acids, alpha hydroxy acids, beta hydroxy acids, gamma hydroxy acids, sugar acids, salts of these, and combinations thereof.

18. The oral care composition of claim 19, wherein the polydentate ligand is selected from oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, citric acid, isocictric acid, aconitic acid, propane-1,2,3-tricarboxylic acid, trimesic acid, pyrophosphate, tripolyphosphate, tetrapolyphosphate, hexametaphosphate, salts of these, and combinations thereof.

19. An oral care product, comprising the composition of claim 1 contained in a translucent or transparent package.
